(19) United States
(12) Patent Application Publication
(10) Pub. No.: US 2024/0201132 A1
(43) Pub. Date: Jun. 20, 2024

(54) IMPROVED DETECTION OF HEMOGLOBIN AND OTHER COMPOUNDS BY ELECTROPHORESIS

(71) Applicant: HEMEX HEALTH, INC., PORTLAND, OR (US)

(72) Inventors: Peter GALEN, Portland, OR (US); David John SAYLER, Portland, OR (US); James Daren BLEDSOE, Portland, OR (US); Muhammad Noman HASAN, Cleveland, OH (US); Joshua King HOYT, Portland, OR (US); Jered WIKANDER, Portland, OR (US); Tyler WITTE, Portland, OR (US); Alireza AVANAKI, Portland, OR (US); Yiyang FEI, Portland, OR (US); Anne ROCHELEAU, Portland, OR (US); Umut Atakan GURKAN, Shaker Heights, OH (US)

(21) Appl. No.: 18/555,955

(22) PCT Filed: Apr. 25, 2022
(86) PCT No.: PCT/US22/71903

§ 371 Date: Oct. 18, 2023

Related U.S. Application Data
(60) Provisional application No. 63/179,118, filed on Apr. 23, 2021.

(51) Int. Cl. G01N 27/447; G01N 33/68

(52) U.S. Cl. CPC G01N 27/44721; G01N 33/68

(57) ABSTRACT

The disclosed methods and systems use multi-spectral imaging of band data in point-of-care devices during active electrophoresis test runs to detect sensitive disease state compounds, such as hemoglobin and its variants and sub-variants. The multi-spectral imaging identifies an imaging wavelength of light that, when emitted towards the electrophoresis strip, optimizes images of the electrophoresis strip that are captured during the test. These optimized images allow for detection of the target compound, particularly at low concentrations and with low limits of detection (LoD).

### CROSS-REFERENCE TO RELATED APPLICATIONS

This application is a 371 filing of International Patent Application No. PCT/US22/71903, filed Apr. 25, 2022 entitled “IMPROVED DETECTION OF HEMOGLOBIN AND OTHER COMPOUNDS BY ELECTROPHORESIS,” which claims priority and benefit from the U.S. Provisional Patent Application 63/179,118, filed Apr. 23, 2022, and titled, “IMPROVED DETECTION OF HEMOGLOBIN AND OTHER COMPOUNDS BY ELECTROPHORESIS,” which is incorporated herein by reference in its entirety for all purposes.

### BACKGROUND

Hemoglobin (Hb) disorders are among the world's most common monogenic diseases Nearly 7% of the world's population carry Hb gene variants. Sickle cell disease (SCD) arises when hemoglobin variant mutations are inherited homozygously (HbSS) or paired with another β-globin gene mutation. Globally, an estimated 400,000 babies are born annually with SCD and 70%-75% are in sub-Saharan Africa (SSA). It is estimated that 50-90% in SSA die by their 5th birthday, 70% of these deaths are preventable. Epidemiological modeling shows that universal screening could save the lives of up to 9,806,000 newborns with SCD by 2050 with 85% born in Sub-Saharan Africa (SSA). Effective management of SCD involves genetic counselling, early diagnosis, and, importantly, newborn screening (NBS).

NBS is a most important public health initiative. SCD NBS performed in centralized laboratories has dramatically dropped SCD mortality in resource-rich countries. NBS requires sensitive detection of certain low level Hb variant from high level Hb variants. For example, among newborns, normal hemoglobin A (HbA) and sickle hemoglobin S (HbS) are at lower levels while high levels fetal hemoglobin (HbF) holds up to 90% of total Hb. In resource-rich countries, standard clinical laboratory technology including high-performance liquid chromatography (HPLC) and isoelectric focusing (IEF) are typically used in testing Hb variant. However, these advanced laboratory techniques require trained personnel and state-of-the-art facilities, which are lacking or in short supply in countries where the prevalence of hemoglobin disorders is the highest.

SCD NBS is challenging in low and middle income countries, where heavy SCD burden exists, due to lack of lab infrastructure and skilled personnel. In a 2019 report, the World Health Organization (WHO) has listed hemoglobin testing as one of the most essential in vitro diagnostic (IVD) tests for primary care use in low and middle income countries. Furthermore, hemoglobin electrophoresis has recently been added to the WHO essential list of IVDs for diagnosing SCD and sickle cell trait. As a result, there is a need in the art for affordable, portable, easy-to-use, accurate, non-capillary flow electrophoresis tests to facilitate decentralized hemoglobin testing in low-resource settings to enable widespread NBS.

### BRIEF DESCRIPTION OF THE DRAWINGS

Non-limiting and non-exhaustive embodiments of the invention are described with reference to the following drawings. In the drawings, like reference numerals refer to like parts throughout the various figures, unless otherwise specified, wherein:

FIG. 1A is a flowchart with steps to detect a compound in a patient sample in a diagnostic device.

FIGS. 1B-1I show example images of electrophoresis strips in various illumination states.

FIG. 2 is an example diagnostic system with multi-spectrum light emission.

FIGS. 3A-3T show example results from detecting and quantifying a compound variant.

FIG. 4 shows scattered plots of comparing the disclosed systems and methods compared to a gold standard HPLC test.

FIGS. 5A and 5B show steps in an algorithm that creates and analyzes a run summary.

FIG. 6 show an example run summary.

### DETAILED DESCRIPTION

The disclosed systems and methods detect and diagnose various disease states including hemoglobinopathies, such as sickle cell disease and trait, thalessemias, and the like. Such disease states as sickle cell disease and trait are important to diagnose early in life so treatment can begin and the effects of disease morbidities are reduced. For example, newborns can be screened for sickle cell disease or trait, especially in regions with populations with a high hereditary percentage of sickle cell carriers. Often these same regions with high populations of sickle cell carriers lack medical resources required to perform conventional gold standard laboratory tests to detect and diagnose the disease state.

The disclosed systems and method use electrophoresis in non-capillary flow electrophoresis that generate band data indicative of compounds present in the patient sample. For example, the non-capillary flow electrophoresis device can be a point-of-care (POC) diagnostic device. The patient sample is often blood but could be other patient biologic material as well. The disclosed electrophoresis systems capture one or more images of the electrophoresis strip on which the patient sample is placed and to which an electric field is applied that separates compounds in the patient sample based on their size and electrical charge. The separation of these compounds produces bands that migrate across the electrophoresis strip during the active test. The disclosed systems and methods capture the one or more images of the electrophoresis strip during the active test and oftentimes throughout the active test in a non-capillary flow electrophoresis device. When the band(s) are produced and an image is desired, light is emitted towards the electrophoresis strip. The light is either absorbed by or fluoresced from the band, which is detectable on the captured image(s).

During the active test, one or more targeted wavelengths of light are emitted towards the electrophoresis strip to produce the desired image(s). The targeted wavelengths can be a range of wavelengths in some examples or a particular color or color range of wavelengths. For example, the emitted light can be in a range of 390-430 nanometers (nm), which is in the ultra-violet (UV) wavelength range. In some examples, a second light emission occurs that could be a different wavelength range than the first light emission, which could be white light or another color of light that produces different absorption or fluorescence characteristics in the image(s) of the band(s) on the electrophoresis strip. The image characteristics produced by the absorption or fluorescence of each wavelength of light emitted towards the electrophoresis strip can vary over time throughout the active test and can vary with different wavelengths of light, camera aperture, etc.

Typically, conventional electrophoresis systems run a complete electrophoresis test on the patient sample on the electrophoresis strip then stain the final strip to cause the bands produced during the test to either absorb or fluoresce in a particular way in response to white light. The absorption or reflection can be controlled by the type of stain(s) applied. However, the staining process is lengthy, expensive, and requires sophisticated laboratory equipment. In low resource regions or remote geographic locations without good access to medical facilities, these expensive laboratory tests are impractical or simply unavailable. Such regions need POC diagnostic devices like the disclosed system to accurately detect and diagnose these disease states.

Many of these diseases have a limit of detection (LoD) that is low, which requires the sophisticated laboratory equipment to run the electrophoresis test with high quality cameras and staining processes available. Even then, there are still patient samples that are too low of a concentration of the target compound to be detected in a conventional system. The LOD of a particular compound or disease indicator (e.g., analyte, antibody, label, etc.) can be a low concentration at which the disease is detectable at an acceptable accuracy level. Traditional POC devices could not perform tests at the LOD required to detect certain disease states, such as sickle cell disease or trait, especially in newborns, and thalassemias for example, because they use images of white light emitted towards a strip that has been stained. These images produce edges that are blurred or their shape or visibility has been sacrificed. Such edge, shape, speed (changes in the band position over time), and visibility detection in the images defines whether the captured image includes a band indicating the target compound. For example, when white light is absorbed or reflected from the bands produced at the end of the electrophoresis test, the band image is hard to detect and, in some cases, has an LOD of 20%, which is not sensitive enough to detect diseases like sickle cell disease and trait or thalessemias without sophisticated imaging systems and dyes or staining processes. For example, the position of a band imaged over time indicates speed of migration of the band on the strip. Such position or speed information obtained from images captured during an active electrophoresis test help to identify low concentrations of compounds that are not consistently present throughout the active run. For example, a compound may not appear during a first or final phase of the active run and is only visible during a middle portion of time in the active run. Taking position or speed data of the band associated with the target compound during the middle portion of the active run can detect the compound while taking an image of the final phase of the run in the conventional technique would miss it entirely. The LOD is lower in the disclosed systems and methods because they use light emitted at the targeted wavelength that produces the highest quality absorption or fluorescence qualities in the compound band.

The wavelength of the light emitted towards the electrophoresis strip is selected based on a maximum or optimal absorption or fluorescence characteristic(s) of the compound of interest. For example, the maximum or optimal characteristics could be the wavelength at which the compound band maximally absorbs light or fluoresces the compound. Compounds differ on their absorption and fluorescence properties or characteristics and respond different to various wavelengths of light. Compounds may not absorb or fluoresce at all in response to emission of certain wavelengths of light while the same compound could produce a clear, intense absorption or fluorescence in response to light emitted towards it at a different wavelength. This “imaging wavelength” is the wavelength at which the highest quality image is produced to analyze for detection of the disease state. In some examples, the imaging wavelength is matched to the target compound of interest based on known empirical data or previous tests performed on bands known to have the target compound.

In other examples, the imaging wavelength can also be matched to the target compound of interest based on matching to a control band. The control band(s) can include the target compound or can exclude the target compound. Their purpose is to serve as a relative point of comparison for images of the other bands produced during the active electrophoresis test. The images of the control bands can be compared in intensity, shape, edge shape, speed or clarity, or any other characteristic that either relates to or discerns from a band with an unknown compound or no compound.

In some example systems, light is emitted towards the electrophoresis strip at multiple wavelengths. The multiple wavelengths can produce different responses in absorption or fluorescence of the bands on the electrophoresis strip. Each of those responses can either validate or provide additional data to each other when the bands images are analyzed. For example, light of a wavelength within a range of ˜410 nm in the UV range of 390-430 nm is emitted at a first time and then a second white light is also emitted at a second time. The first time and the second time are temporally spaced apart any suitable amount of time. In another example, the emission occurs at the same time.

In the example in which the light at the imaging wavelength is emitted towards the strip at the first time followed by the light emission at the second wavelength at the second time, when using the disclosed systems for detection of sickle cell disease and anemia, both white light and UV produce images of hemoglobin and marker: UV provides a mode of detection of low levels of sickle cell disease or trait, for example, which has an LOD of ˜4%. In this case, the white light image is used for to separate the marker, allowing for marker-only tracking (no hemoglobin is visible in the red channel of white images) and for calculating the hemoglobin to marker concentration ratio.

Bands with low concentrations of hemoglobin are only visible in UV since hemoglobin absorbs 410 nm, for example, much more than it absorbs white light. For sickle cell disease, for example, the targeted UV light emission allows the sickle cell (hemoglobin-S or HbS) band to absorb light at a detectable level in the captured image. Other wavelengths of light cannot produce the same level or response profile of absorbance of the HbS band.

In detecting HbS in newborns, the required LOD is low—4% or less—and can be masked by presence of fetal Hb or HbF, which has a high or 90% concentration at birth and its concentration is reduced in the first few months of life. Detecting HbS is more difficult in the presence of HbF, especially at early age with high concentrations of HbF. Because of the high concentration of HbF, newborns are particularly hard patients in which to detect HbS.

In some examples, the system captures multiples images of the electrophoresis strip during the active test. These multiple images are combined to produce an enhanced image of the target compound band. To create the enhanced image, the target compound band images can be overlaid to ensure accurate band detection or could be compared to each other to validate data, ensure the edge of the band or the shape is consistent with a compound profile or is consistently developing over time during the test in an expected manner to match it with a target compound profile of the same development or compare it to empirical or threshold data. The enhanced image can be output to a display for a user to visually analyze, in some examples, or could be stored.

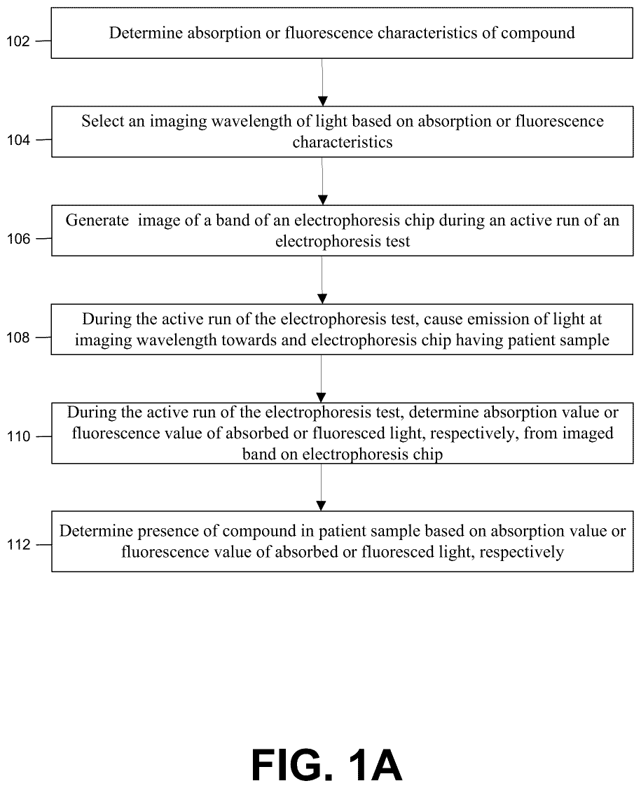

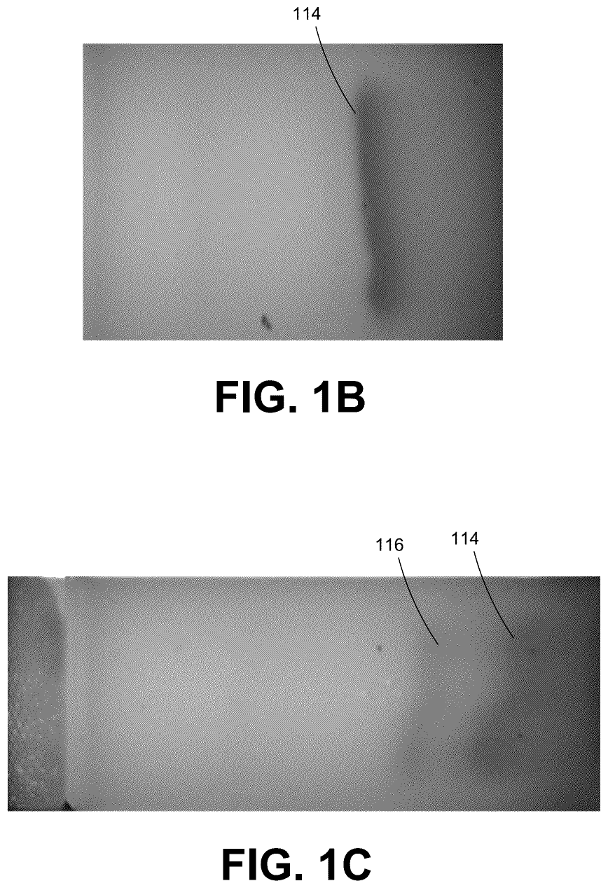

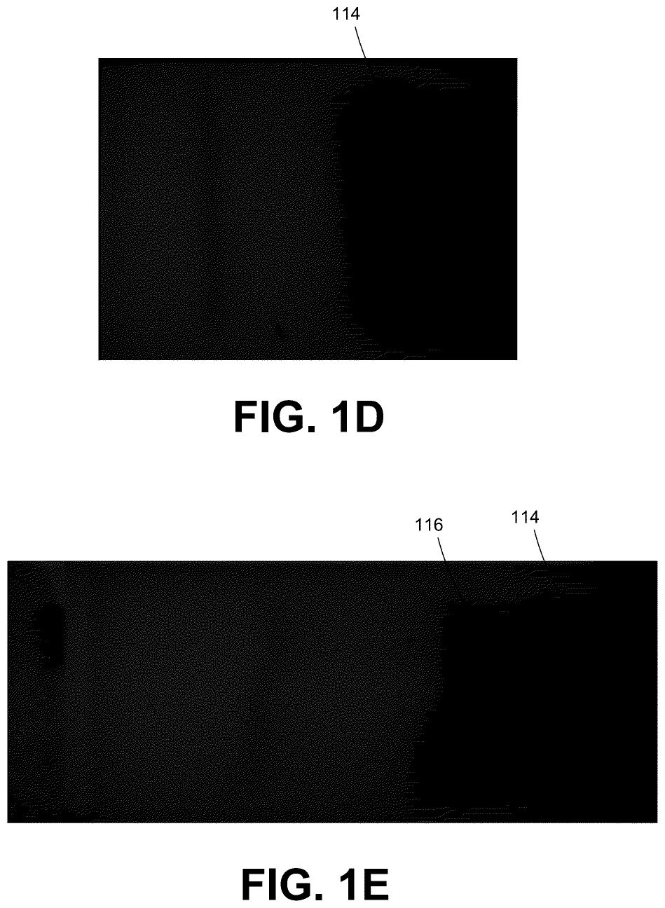

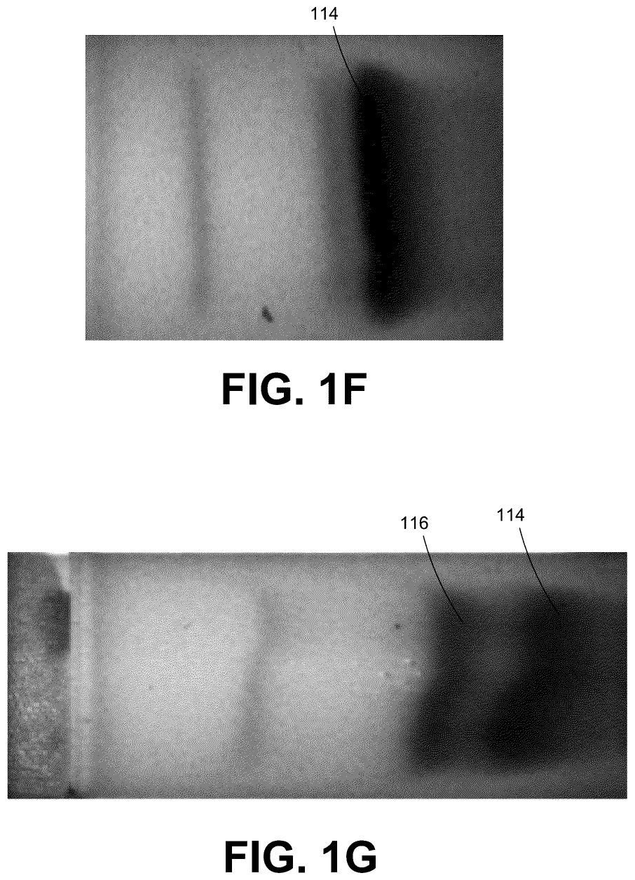

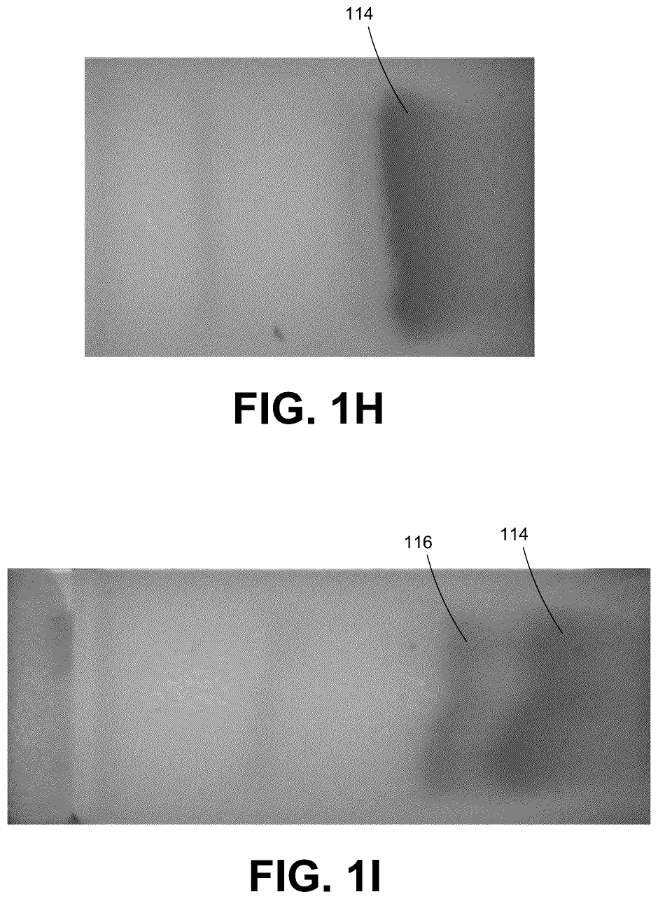

Multiple images can provide false colors by combining the images of the light emitted in overlapped wavelength ranges. For example, a UV image can be combined with a white image (e.g., replacing green and blue channels of white with information coming from UV image adapted by histogram matching) to enhance the visibility of the faint blood bands while preserving the familiar appearance of a white-lit image. FIGS. 1B and 1C show captured images of an image of an electrophoresis strip illuminated with white light that shows a control band 114 and a target compound band 116. FIGS. 1D and 1E show captured images of the control band 114 and a target compound band 116 the same electrophoresis strip illuminated with UV light at 410 nm in this example. FIGS. 1F and 1G show captured images of the control band 114 and a target compound band 116 the same electrophoresis strip illuminated with greyscale UV light. FIGS. 1H and 1I show composite images of the control band 114 and the target compound band 116 the same electrophoresis strip illuminated with UV light at 410 nm in this example. The composite images shown in FIGS. 1H and 1I combine the image data from the white images shown in FIGS. 1B and 1C and the UV light images shown in FIGS. 1D and 1E to create images that are intelligible to the human user to appear similar to a stained image produced by the conventional imaging technique.

In some examples, the disclosed systems and methods can also create a single representation that includes all the band information—the detected characteristics of each imaged band—from all images (or multiple images) captured during the active electrophoresis test. The single representation is a run summary of imaged results of the target compound band throughout the entire the electrophoresis test. Creating the run summary diagram is the first step of the speed profiling algorithm that interprets the decomposition of the patient sample content to different Hb variants based on their electrophoresis speed throughout the active test.

FIG. 1 shows a flowchart with steps for detecting a compound in a patient sample 100. This process can be detected in the disclosed systems through an integrated algorithm that process the received data. The algorithm can also receive external data, such as from a data store or other source, to help in the data analysis. The algorithm determines absorption or fluorescence characteristics of a target compound 102. The absorption or fluorescence characteristics relate to image characteristics that are produced when light is emitted towards a band that is imaged during the electrophoresis test. For example, absorption characteristics occur when light is absorbed by the band while fluorescence characteristics occurs when light is fluoresced from the band. The absorption or fluorescence characteristics can be determined by empirical data previously collected on known compounds or by comparison of image characteristics to a control band of a known compound, for example. The absorption or fluorescence characteristics can be determined by the system or could be received from an external source.

The method then selects an imaging wavelength of light based on the absorption or fluorescence characteristics of the compound 104. The imaging wavelength is the wavelength of light that produces the optimal image characteristics to analyze to detect the compound in the patient sample. Typically, the imaging wavelength causes the absorption or fluorescence characteristics to be enhanced compared to the imaged band's response to light of a wavelength other than the imaging wavelength. The enhanced image typically allows for detection of the band at a lower concentration of the compound in the patient sample or can produce an image that defines the band edge or shape of the band with greater clarity to quantify the compound or to otherwise give detect data relating to the compound. For example, using images captured using UV light, it was observed that the shape of the blood band concentration signal peaks are better modelled by skewed generalized gaussian distribution rather than standard gaussian. Switching to skewed generalized gaussians improved quantification accuracy. Finding that a band is sharp and narrow or diffuse and wide can also be used to determine a test failure since a very wide diffuse band can indicate an issue that renders the results invalid.

The method then selects an imaging wavelength of light based on the absorption or fluorescence characteristics of the compound 106. The absorption or fluorescence characteristics of the compound are a compound profile that optimizes the image produced when the band is imaged throughout the active test. The determination of the absorption or fluorescence characteristics of the compound 104 and the selection of the imaging wavelength of light 106 can be performed by a remote computing device, server, or system or can be integrated into any of the disclosed systems. The disclosed method then generates an image of a band on an electrophoresis strip during an active run of an electrophoresis test 108. The image can be captured by an imaging device, such as an optical imaging device like a camera, which captures an image of the electrophoresis strip. In some examples, the method captures multiple images timed periodically, randomly, manually, or in a particular sequence or on a specific schedule throughout the active run of the electrophoresis test.

The method also causes emission of light at the imaging wavelength towards an electrophoresis strip with the patient sample. As mentioned above, this occurs during the active electrophoresis test. The light can be emitted by any source that is either integrated within the system or external to the system. The method then determines an absorption characteristic or a fluorescence characteristic of absorbed or fluoresced light, as the case may be from the selected compound, which occurs during the active test 112. The absorption characteristic or fluorescence characteristic can be a relative or absolute value, for example. As explained above, the selected compound may either absorb or fluoresce light at the imaging wavelength. The method then determines the presence of the compound in the patient sample based on the absorption characteristic or the fluorescence characteristic of the absorbed or fluoresced light 114. The compound type or a variant or sub-variant of the compound can be determined from the absorption characteristic, the fluorescence characteristic, the shape of the band, the edge of the band, the position of the band, the speed of the band, or other defining band characteristics. The captured image(s) can be compared against empirical data or compared to image characteristics of a control band in the same active test or other analysis of the data. The data relating to determining the presence of the compound can optionally be output to a data store, an external device, a display, or the like.

Various system parameters can be adjusted in some examples that enhance or provide a different perspective on the captured image(s). For example, the intensity of the light emitted towards the electrophoresis strip is adjusted from a first intensity to a second intensity. The image characteristics of the image(s) captured of the electrophoresis strip when the light at the first intensity is emitted is different than the image characteristics of the image(s) captured of the electrophoresis strip when the light at the second intensity is emitted. That difference value can be calculated and used to determine presence of the compound. If the difference value exceeds a threshold, for example, then the compound is determined to be present.

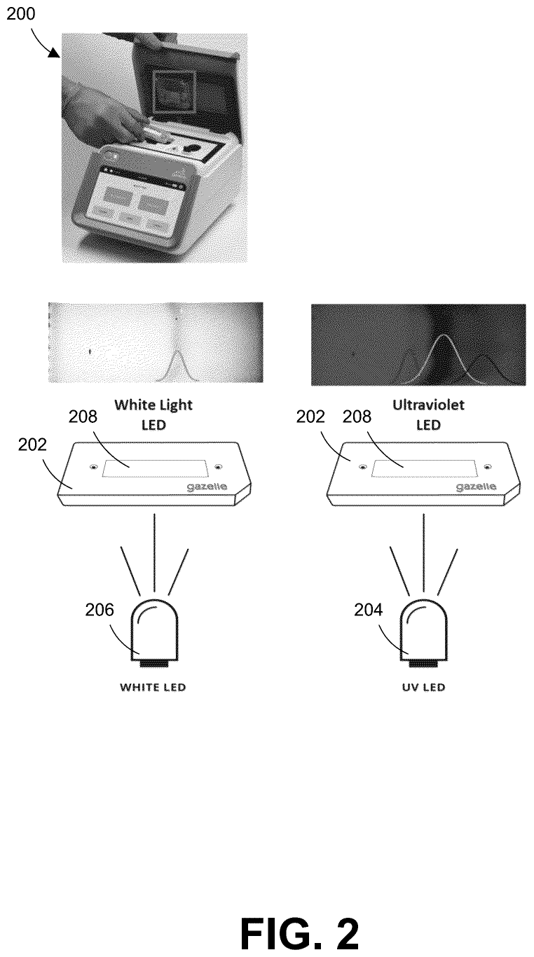

Turning now to FIG. 2, the disclosed non-capillary electrophoresis system detects a compound in a patient sample. The system can separate, image, and track the target compound and its variants and sub-variants in real-time during an active electrophoresis test under multi-spectrum light emission. The non-capillary electrophoresis system includes a reader 200 that has an integrated circuitry (not shown) to apply voltage to an inserted electrophoresis strip 202 in a standard manner of conducting an electrophoresis test. During the active test, the reader 200, activates two light emissions, in this example, which include light emission in the UV range 204 and a second white light emission 206. FIG. 2 shows these two light emissions 204, 206 as separate light sources although they can be the same light source with filters applied to produce the UV light 204 or can be integrated into a single light source capable of emitting white light and UV light. The UV light 204 and the white light 206 can be emitted at the same time or at different times, depending on the target compound response characteristics.

In the example shown in FIG. 2, the target compound is hemoglobin among other biomolecules. The hemoglobin variants can be separated based on their charge-to-mass ratio when exposed to an electric field in the presence of a carrier substrate, which is the cellulose acetate paper 208 in the electrophoresis strip 202 in this example. In this example, the patient sample is blood and is obtained from a finger prick, which typically yields about 25 μL per drop. The sampled blood is prepared by mixing and lysing it with a standard calibrator solution. The prepared same contains lysed blood and standard calibrator, which is loaded onto the electrophoresis strip for electrophoresis. In this example, a Tris/Borate/EDTA (TBE) buffer is used to provide the necessary ions for electrical conductivity at a pH of 8.4 in the cellulose acetate paper. The pH induced net negative charges of the hemoglobin variants and the standard calibrator molecules cause them to travel from the negative to the positive electrode when placed in an electric field. The electric mobility differences of among various hemoglobin phenotypes allow separation of the hemoglobin variants. Each variant is identified by its electric mobility differences in the images captured of the strip during and throughout the active electrophoresis test. In this example, the separated hemoglobin variants are imaged under both white light 206 and UV light 204.

Hemoglobin at high concentrations can be detected by both white light and UV light. The acquired data under white light field demonstrates natural red color of hemoglobin. The images captured during the UV light emission is used for detection of low concentration hemoglobin variants and for quantification of individual Hb variants. For example, the shape of the band correlates to a quantification of the concentration of the compound in the patient sample. Data acquired under UV light has enhanced LOD (lower LOD) and higher signal to background noise ratio than white light filed data, which is also shown in FIG. 2 and described in more detail below. Combining both white light and UV light image data allow the disclosed systems to track, detect, identify, and quantify electrophoretically separated low concentrations of Hb variants.

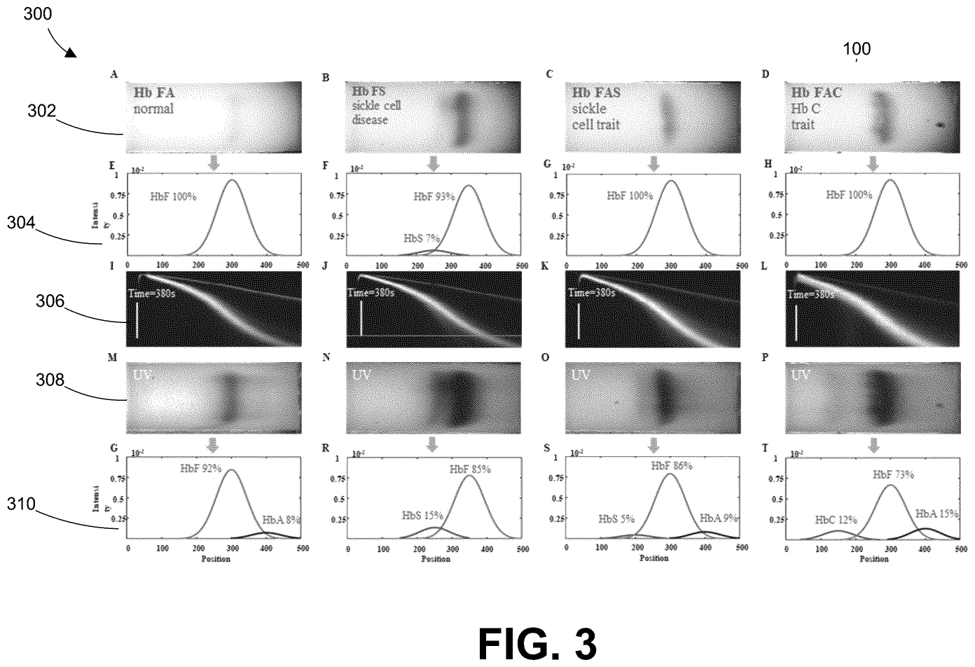

Turning now to FIGS. 3A-3T, the target compound detected and quantified in this example is hemoglobin 300. As discussed above, any other compound can be detected or quantified using the disclosed systems and methods. The first row 302 shows images captured with white light. The second row 304 shows electropherograms generated based on the white light image. The third row 306 illustrates 2D representation of the disclosed systems and methods that use multi-spectral techniques to detect compounds. The 2D representations are space-time plots—a run summary, creation and usage of which are described later—of band migration in a UV light imaging mode. The fourth row 308 shows representative images captured in the ultraviolet light imaging mode. The fifth row 310 shows electropherograms generated based on the UV light image using the data analysis algorithm. The fifth row 310 shows Hb types and fractions (%) were identified using the multispectral test for a sample with normal HbFA (Healthy newborn), HbFS (Newborn with sickle cell disease), HbFAS (Newborn with sickle cell disease trait), and HbFAC (hemoglobin C disease), respectively. UV imaging enabled identification and quantification of low concentration Hb variants with higher sensitivity (I-T) compared to white light imaging mode (A-H). The UV imaging can be coupled with the machine learning algorithm that discerns between data sets at continually smaller differences to detect very small changes in images over time.

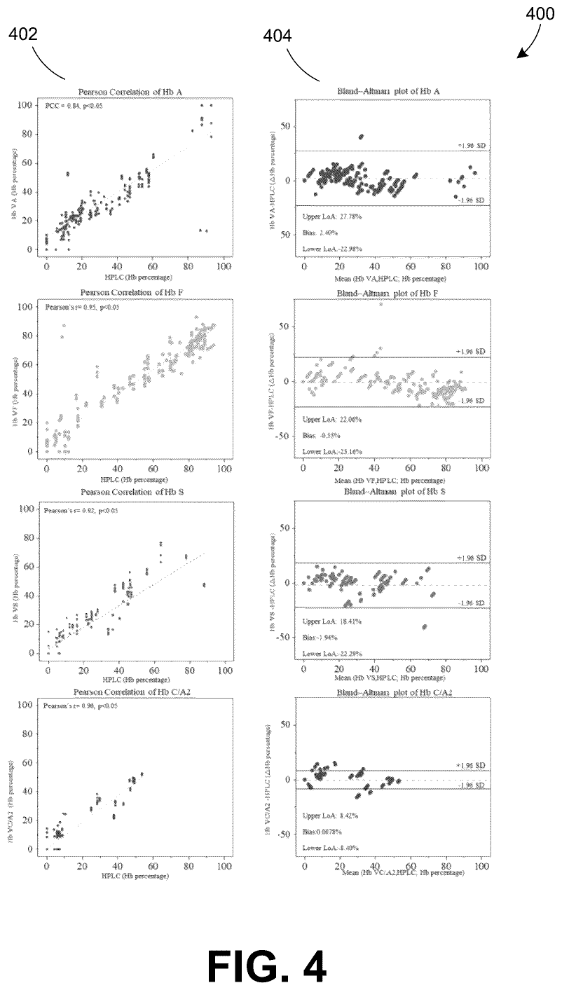

FIG. 4 shows performance plots that are based on space-time plots developed according to the band migration captured under UV light for various Hb types, variants, and sub-variants. The scatter plots include determined levels (y axis) versus the Hb levels reported by the HPLC within 226 tested samples with a variety of Hb variants including HbA, HbF, HbS, and Hb C/E/A2. The scattered plot demonstrates Pearson correlation coefficients (PCC) of 0.84, p<0.05 for HbA; PCC=0.95, p<0.05 for HbF; PCC=0.92, p<0.05 for HbS; and PCC=0.96, p<0.05 for Hb C/E/A2. These results reveal strong association between disclosed multi-spectral technique's determination of the Hb variant levels and the laboratory HPLC technique's reported Hb variant levels. The Bland-Altman analysis 404 showed the disclosed multi-spectral technique determines blood Hb variant levels with biases of 2.4% (95% limits of agreement (LOA): 25.4%) for HbA, −0.55% (95% LOA: 22.6%) for HbF, −1.94% (95% LOA: 20.4%) for HbS, and 0.0078% (95% LOA: 8.41%) for Hb C/E/A2.

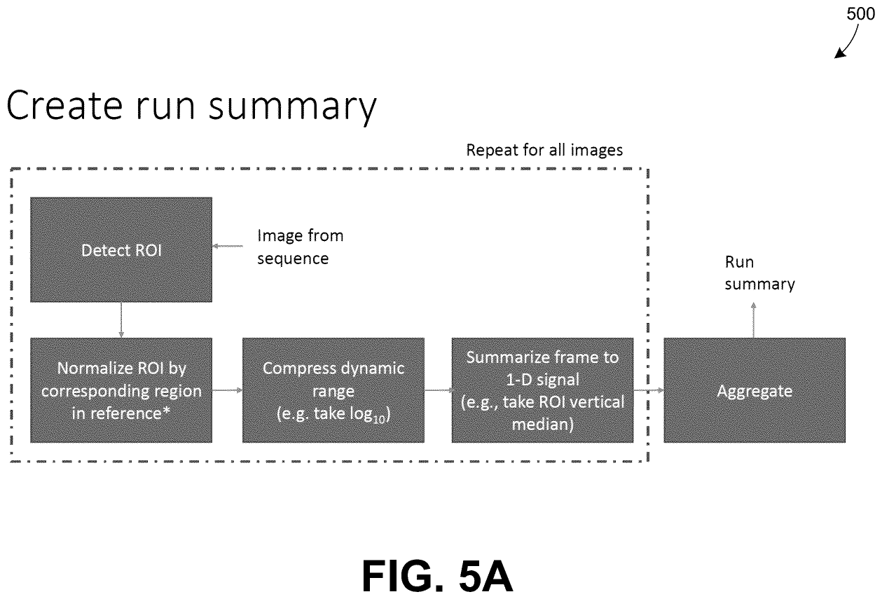

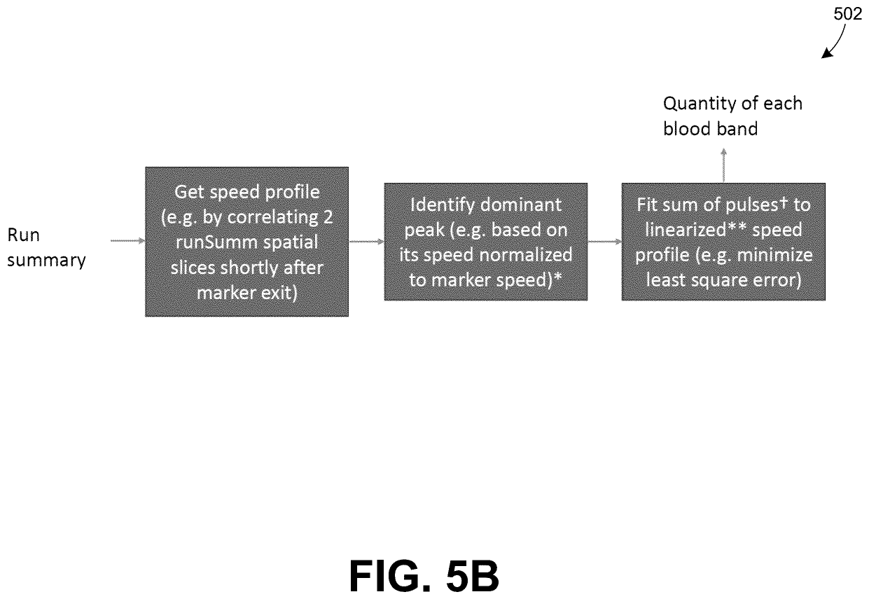

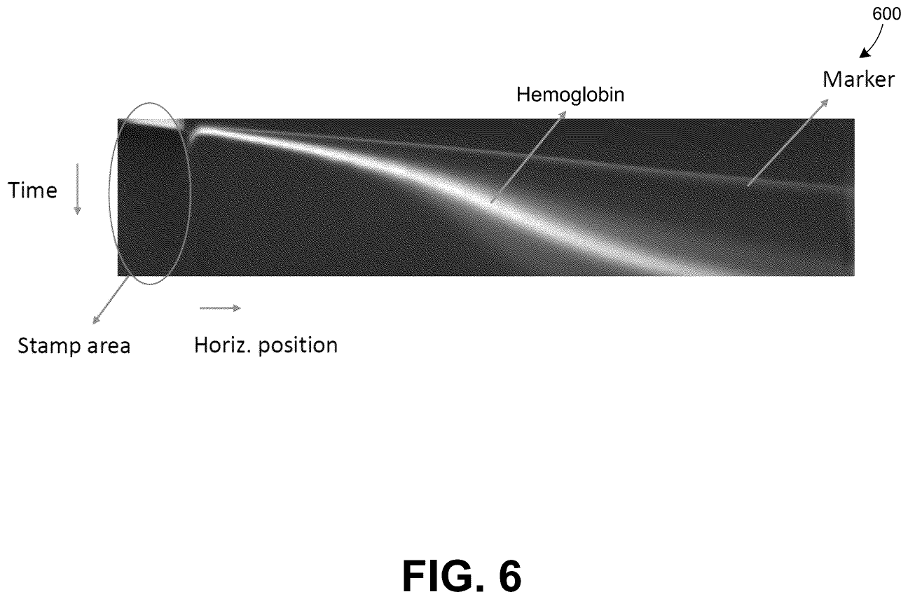

FIGS. 5A and 5B show steps of an algorithm that creates a run summary, then analyzes it. Characteristics of each imaged band are detected and those characteristics are compiled into a single image from all images or multiple images from a portion of all of the images. The compiled images are captured during the active electrophoresis test. The run summary is a single representation of imaged results of the target compound band throughout the entire or a portion of the electrophoresis test. The run summary is part of speed profiling algorithm that indicates decomposition of the patient sample content to different Hb variants based on their electrophoresis speed throughout the active test. In FIG. 5A, run summary creation is explained. Run summary is a diagram that describes the (horizontal) spatial distributions of hemoglobin and marker in each frame. This is done by normalizing each frame to a reference frame (e.g., one without any blood or marker) to calculate an absorption map, which is then collapsed along its vertical dimension (e.g., by taking vertical average or median on whole frame or its parts e.g., horizontal stripes). This will create one raster/horizontal line of the run summary, showing horizontal hemoglobin/marker distribution corresponding to that frame. The run summary diagram is made of such raster lines, each coming from one frame, in chronological order, showing hemoglobin/marker distribution over time. A marker-only run summary, e.g., for easy marker tracking, can be created using frames captured under red light, or the blue channel of white-light captures. In short, from the run summary diagram one can see how much hemoglobin of which type is moving at what speed: the speed of the specific hemoglobin type trace is its slope in the run summary. For example, in FIG. 6, faster hemoglobin appears more horizontal (stationary hemoglobin would appear vertical).

In FIG. 5B, a run summary diagram is shown that derives the types and amounts of Hb variants present in the sample. The horizontal hemoglobin distributions are correlated in two different frames shortly after marker exit (i.e., marker is no longer visible on the cartridge). This timing is apt because it provides a good separation between Hb variants before the hemoglobin bands widen and fade away. The result of this operation is a speed profile: the concentration of hemoglobin moving at any given speed (as a fraction of marker speed). The Hb variants are then identified based on speed (e.g., HbA is almost as fast as the marker and HbC is nearly stationary later in the run) and behavior (e.g., HbA tends to speed up later in the run while HbS speed does not change). Moreover, the leading and trailing edges of hemoglobin bands before marker exit can also be used in variant identification (e.g., HbA moves faster than HbF, and HbS slower than HbF). Quantification of each Hb variant is achieved by fitting a parametric model (e.g., sum of skewed-generalized gaussians, one for each hemoglobin band identified) to the observed hemoglobin distribution.

FIG. 6 shows an example run summary 600 with time (and hence frame number) increasing down the y-axis and position on the electrophoresis strip or paper increasing along the x-axis. In the example shown in FIG. 6, the run summary 600 is a compilation of 120 frames at 4 second intervals (each correspond to the one horizontal raster line on the run summary) taken throughout the duration of the electrophoresis test. The dominant blood band is HbF (darker means more absorption, which means more hemoglobin). This sample also contains some HbA (running faster, thus to the right of HbF), and some HbS (moving slower, thus to the left of HbF).

The subject matter of embodiments disclosed herein is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

What is claimed is:

1. A method of detecting a compound in a patient sample in a point-of-care diagnostic device, comprising:
determining absorption or fluorescence characteristics of the compound;
selecting an imaging wavelength of light based on the absorption or fluorescence characteristics;
during the active run of the electrophoresis test, causing emission of light at the imaging wavelength and at a second wavelength towards an electrophoresis strip having the patient sample;
generating an image of a band on an electrophoresis strip during an active run of an electrophoresis test;
during the active run of the electrophoresis test, determining an absorption characteristic or a fluorescence characteristic of absorbed or fluoresced light, respectively, from the imaged band on the electrophoresis strip; and
determining presence of the compound in the patient sample based on the absorption characteristic or the fluorescence characteristic of the absorbed or fluoresced light, respectively.

2. The method of claim 1, wherein the compound has a limit of detection (LoD) in the patient sample when imaged during emission of the light at the imaging wavelength that is lower than an LoD of the compound when imaged during emission of white light or light at a wavelength other than the imaging wavelength.

3. The method of claim 1, further comprising determining a variant type or a sub-variant type of the compound in the patient sample based on the absorption characteristic, the fluorescence characteristic, a position over time of a band indicative of the variant type or the sub-variant type of the compound, or a shape of the band.

4. The method of claim 1, wherein selecting the imaging wavelength of light based on the absorption characteristics includes selecting a wavelength of maximum absorption or fluoresce of the compound.

5. The method of claim 1, wherein selecting the imaging wavelength of light based on the absorption characteristics includes selecting a wavelength of maximum absorption or fluoresce of the compound of within a range of 390-430 nanometers (nm).

6. The method of claim 1, wherein during the active run of the electrophoresis test, causing emission of light at the imaging wavelength at a first time during the active run and causing emission of light at the second wavelength at a second time during the active run.

7. The method of claim 1, further comprising:
determining a shape of the band on the electrophoresis strip; and
quantifying the compound based on a characteristic of the shape and location of the band.

8. The method of claim 1, further comprising:
adjusting an intensity of the emission of the light at the imaging wavelength from a first intensity to a second intensity; and
determining presence of the compound in the patient sample based on a difference between the absorption characteristic or the fluorescence characteristic of the absorbed or fluoresced light, respectively, at the first intensity and the second intensity.

9. The method of claim 1, further comprising outputting data indicating presence of the compound in the patient sample.

10. The method of claim 1, further comprising:
generating multiple images of the band on the electrophoresis strip during the active run of the electrophoresis test;
determining absorption characteristics or fluorescence characteristics of the absorbed or fluoresced light, respectively, from the multiple images of the band on the electrophoresis strip; and
determining presence of the compound in the patient sample based on the absorption characteristics or the fluorescence characteristics of the absorbed or fluoresced light, respectively.

11. The method of claim 10, further comprising:
during the active run of the electrophoresis test, causing emission of white light towards the electrophoresis strip having the patient sample;
during the active run of the electrophoresis test, generating a first of the multiple images of the band on the electrophoresis strip during the emission of light at the imaging wavelength; and
generating a second of the multiple images of the band on the electrophoresis strip during the emission of white light.

12. The method of claim 11, further comprising generating a compiled image that overlays the first of the multiple images and the second of the multiple images.

13. The method of claim 1, wherein causing emission of light within a range of wavelengths that includes the imaging wavelength, the light emitted towards the electrophoresis strip having the patient sample, and further comprising applying a filter to the emitted light to limit the light emitted towards the band to be the light with the imaging wavelength.

14. A system for detecting a compound in a patient sample in a point-of-care diagnostic device, comprising:
a processor configured to:
receive absorption characteristics of the compound;
select an imaging wavelength of light based on the absorption characteristics;
during the active run of the electrophoresis test, generate an instruction to cause emission of light at the imaging wavelength and at a second wavelength towards an electrophoresis strip having the patient sample;
generate an image of a band on an electrophoresis strip during an active run of an electrophoresis test;
during the active run of the electrophoresis test, determine an absorption characteristic or a fluorescence characteristic of absorbed or fluoresced light, respectively, from the imaged band on the electrophoresis strip; and
determine presence of the compound in the patient sample based on the absorption characteristic or the fluorescence characteristic of the absorbed or fluoresced light, respectively; and
an output configured to output data that indicates the presence of the compound in the patient sample.

15. The system of claim 14, wherein the compound has a limit of detection (LoD) in the patient sample when imaged during emission of the light at the imaging wavelength that is lower than an LoD of the compound when imaged during emission of white light or light at a wavelength other than the imaging wavelength.

16. The system of claim 14, wherein the processor is further configured to determine a variant type or sub-variant type of the compound in the patient sample based on the absorption characteristic, the fluorescence characteristic, or a shape of the band.

17. The system of claim 14, wherein the processor is further configured to select the imaging wavelength of light based on the absorption characteristics of selecting a wavelength of maximum absorption or fluoresce of the compound.

18. The system of claim 14, wherein the processor is further configured to select the imaging wavelength of light based on the absorption characteristics of selecting a wavelength of maximum absorption or fluoresce of the compound of in a range of 390-430 nanometers (nm).

19. The system of claim 14, wherein the processor is further configured to generate an instruction to cause emission of light at the imaging wavelength at a first time and to generate an instruction to cause emission of light at the second wavelength at a second time.

20. The system of claim 14, wherein the processor is further configured to:
determine a shape of the band on the electrophoresis strip; and
quantify the compound based on a characteristic of the shape of the band.

21. The system of claim 14, wherein the processor is further configured to:
adjust an intensity of the emission of the light at the imaging wavelength from a first intensity to a second intensity; and
determine presence of the compound in the patient sample based on a difference between the absorption characteristic or the fluorescence characteristic of the absorbed or fluoresced light, respectively, at the first intensity and the second intensity.

22. The system of claim 14, wherein the processor is further configured to output data indicating presence of the compound in the patient sample.

23. The system of claim 14, wherein the processor is further configured to:
generate multiple images of the band on the electrophoresis strip during the active run of the electrophoresis test;
determine absorption characteristics or fluorescence characteristics of the absorbed or fluoresced light, respectively, from the multiple images of the band on the electrophoresis strip; and
determine presence of the compound in the patient sample based on the absorption characteristics or the fluorescence characteristics of the absorbed or fluoresced light, respectively.

24. The system of claim 22, wherein the processor is further configured to:
during the active run of the electrophoresis test, cause emission of white light towards the electrophoresis strip having the patient sample;
during the active run of the electrophoresis test, generate a first of the multiple images of the band on the electrophoresis strip during the emission of light at the imaging wavelength; and
generate a second of the multiple images of the band on the electrophoresis strip during the emission of white light.

25. The system of claim 23, wherein the processor is further configured to generate a compiled image that overlays the first of the multiple images and the second of the multiple images.

26. The system of claim 14, wherein the processor is further configured to;
emit light within a range of wavelengths that includes the imaging wavelength, the light emitted towards the electrophoresis strip having the patient sample; and
apply a filter to the emitted light to limit the light emitted towards the band to be within the imaging wavelength.

27. The system of claim 14, wherein the compound includes an analyte, an antibody, or a label.
